(19) United States
(12) Patent Application Publication
(10) Pub. No.: US 2007/0264211 A1
(43) Pub. Date: Nov. 15, 2007

(54) Composition Comprising Sodium Silicate for Use in the Treatment of Dry Skin Conditions

(75) Inventor: Mark Randle, Newcastle-upon-Tyne (GB)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Dermasalve Limited, Newcastle-Upon-Tyne (GB)

(21) Appl. No.: 10/592,447

(22) PCT Filed: Mar. 8, 2005
(86) PCT No.: PCT/GB05/00884

§ 371 Date: Jun. 19, 2007

(30) Foreign Application Priority Data
Mar. 8, 2004 (GB) 0405017.5

(51) Int. Cl. A61K 33/00; A61P 17/06; A61P 17/10; A61Q 17/04; A61Q 19/00

(52) U.S. Cl. 424/60; 424/600

(57) ABSTRACT

A composition contains sodium silicate and is used in the treatment of dry skin conditions, inter alia psoariasis, dermatitis and eczema. The composition is formulated into creams, ointments and sprays, each formulation being adapted to the intended skin application area.

### FIELD OF THE INVENTION

This invention relates to compositions for use in the treatment of dry skin conditions, and in particular to compositions containing sodium silicate.

### BACKGROUND OF THE INVENTION

Many individuals suffer from dry skin conditions causing irritation of the skin, for example eczema, dermatitis, psoariasis, acne, acne rosacea, cradle cap, dermatophyte infections (nail infections), external vaginal thrush, facial malar rash due to systemic lupus erythmatosis, chapped or otherwise damaged skin, etc (and in the context of this application the term “dry skin condition” shall be taken to include dry skin or damaged nails resulting from these conditions or any condition resulting in dry, damaged or chapped skin). These conditions are unsightly, can cause itching, and in some circumstances discomfort

Many treatments are available for the above-mentioned conditions, some of which are more successful than others.

The present invention is concerned with compositions containing sodium silicate. Sodium silicate is known for use in the treatment of wounds following surgery. The invention however is concerned with the use of low concentrations of sodium silicate in compositions for topical application to an area of dry-skin.

Typically, products developed to treat skin irritations are sold in one or two different applications. The problem with such practice is that an individual suffering from a dry skin condition needs to use skin products for purposes which the treatment compositions are not suitable. The invention therefore concerns a range of products for application to, or use in relation to, the skin.

### SUMMARY OF THE INVENTION

One aspect of the invention provides a composition as specified in Claim 1.

Another aspect of the invention relates to the use of a composition comprising sodium silicate as specified in Claim 2.

Preferred aspects of the invention are specified in the claims dependent on Claim 1 and Claim 2.

The creams, ointments and sprays of the invention provide sufferers of dry skin conditions with a treatment that is particularly effective. Furthermore, the treatment can be incorporated into a number of different delivery products, each for application to the skin. By providing such a range of products, each one individually formulated to take account of the skin area to which it is to be applied, sufferers of dry skin conditions can use all the types of skin product that they might wish to use without fear of those products irritating their skin conditions.

### EXAMPLE 1

In order to assess the efficacy of compositions for the treatment of dry skin conditions, a trial was conducted amongst a statistically significant population.

In the trial, participants followed a program using a product containing sodium silicate as the active skin treatment ingredient.

Participants applied the product to the dry skin area during washing/bathing. Following drying with a towel, participants worked more of the product into the skin areas affected by a dry-skin condition. This latter layer of the product was not removed by rinsing, but rather is left to dry on the skin The process of applying the product directly to the dry-skin area (without further washing of bathing) is repeated 2 or 3 times daily.

During the trial participants were required to use no other skin products, and not to use any additives in their laundry, such as fabric conditioner.

Results of the trial are illustrated in tables 1 to 3, each table illustrating the results for sufferers of different dry-skin conditions.

Results

TABLE 1
Psoariasis sufferers summary of efficacy feedback questionaires

| | Total number | Degree of improvement in skin condition | | |
|---|---|---|---|---|
| Age Group | in age group | Slight | Major | clearance of condition |
| <15 | 7 | 0 | 7 | 0 |
| 16-30 | 2 | 2 | 0 | 0 |
| 31-45 | 17 | 11 | 6 | 0 |
| 46-59 | 62 | 14 | 44 | 4 |
| 60+ | 41 | 10 | 16 | 5 |
| Total no.s | 129 | 37 | 73 | 9 |

24 individuals reported no improvement of their skin condition

based upon these results 83.5% of Psoadiasis sufferers in this survey reported and fed back to us subjective evidence of improvement in their condition. 53% of responders reported either a major improvement or a complete clearance of their skin condition.

TABLE 2
Eczema sufferers summary of efficacy feedback questionaires

| | Total number | Degree of improvement in skin condition | | |
|---|---|---|---|---|
| Age Group | in age group | Slight | Major | clearance of condition |
| <15 | 28 | 5 | 17 | 6 |
| 16-30 | 4 | 0 | 4 | 0 |
| 31-45 | 9 | 1 | 3 | 5 |
| 46-59 | 0 | 0 | 0 | 0 |
| 60+ | 15 | 1 | 6 | 8 |
| Total no.s | 56 | 7 | 30 | 19 |

12 individuals reported no improvement of their skin condition

based upon these results 83% of eczema sufferers in thiis survey reported and fed back to us subjective evidence of improvement in their condition. 72% of responders reported either a major improvement or a complete clearance of their skin condition.

TABLE 3
Dermatitis and Acne Rosacea

| | Total number | Degree of improvement in skin condition | | |
|---|---|---|---|---|
| Age Group | in age group | Slight | Major | clearance of condition |
| <15 | 0 | 0 | 0 | 0 |
| 16-30 | 0 | 0 | 0 | 0 |
| 31-45 | 1 | 0 | 2 | 1 |
| 46-59 | 7 | 1 | 6 | 0 |
| 60+ | 2 | 0 | 2 | 0 |
| Total no.s | 12 | 1 | 10 | 1 |

0 individuals reported no improvement of their skin condition

Whilst the group size is small, the results evidence successful treatment of skin disorders other than psoariasis and eczema.

### EXAMPLE 2

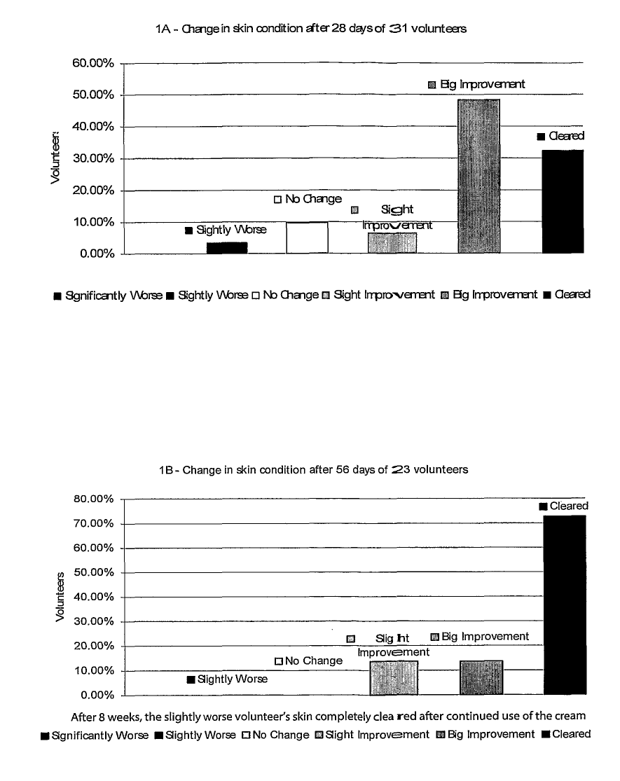

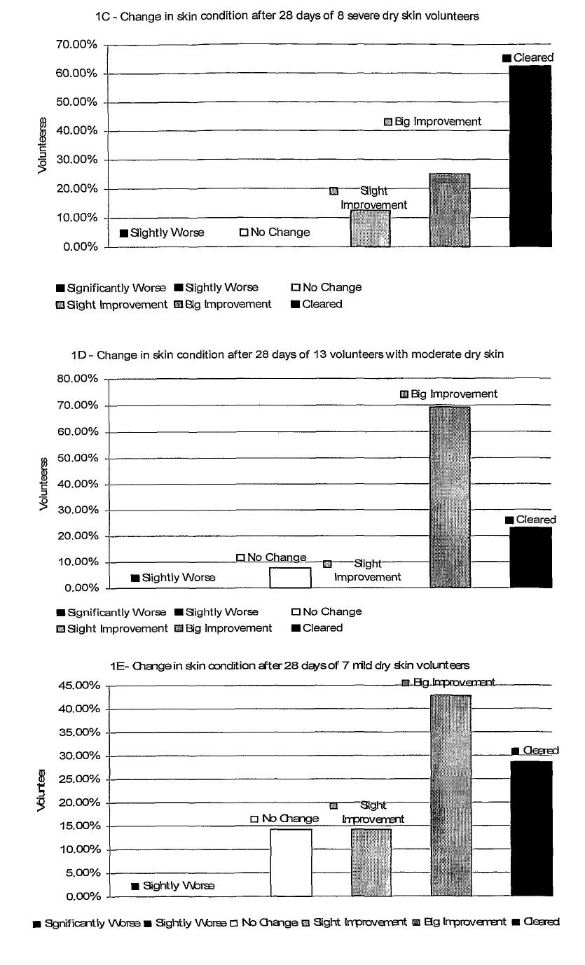

A cream formulation as set out in table 5 below was trialled by a statistically significant group of individuals. FIGS. 1A to 1F illustrate the affects of the cream.

In FIG. 1A, 31 volunteers reported the change in their skin condition after 28 days of applying the cream on to areas of dry skin or infected nails. 32% reported the condition to have cleared, 48% reported a big improvement in their condition, 7% a slight improvement, 10% no change, and 3% slightly worse, i.e. 87% of participants reported some improvement and only 3% the condition becoming slightly worse.

In FIG. 1B, 23 volunteers reported the change in their slin condition after 23 days of applying the cream on to areas of dry skin or infected nails. 72% reported the condition to have cleared, 14% reported a big improvement in their condition, 14% a slight improvement, i.e. 100% of participants reported some improvement.

In FIG. 1C, 8 volunteers each having severe dry skin conditions reported the change in their skin condition after 28 days of applying the cream on to areas of dry skin. 62% reported the condition to have cleared, 26% reported a big improvement in their condition, 12% a slight improvement, i.e. 100% of participants reported some impro In FIG. 1D, 13 volunteers each having moderate dry skin conditions reported the change in their skin condition after 28 days of applying the cream on to areas of dry skin. 23% reported the condition to have cleared, 69% reported a big improvement in their condition, 8% no change, i.e. 92% of participants reported some improvement.

In FIG. 1D, 13 volunteers each having moderate dry skin conditions reported the change in their skin condition after 28 days of applying the cream on to areas of dry skin. 23% reported the condition to have cleared, 69% reported a big improvement in their condition, 8% no change, i.e. 92% of participants reported some improvement

In FIG. 1E, 7 volunteers each having mild dry skin conditions reported the change in their skin condition after 28 days of applying the cream on to areas of dry skin. 29% reported the condition to have cleared, 43% reported a big improvement in their condition, 14% a slight improvement, 14% no change, i.e. 86% of participants reported some improvement.

In FIG. 1F, 5 child volunteers reported the change in their skin condition after 28 days of applying the cream on to areas of dry skin. 20% reported the condition to have cleared, 60% reported a big improvement in their condition, 20% slightly worse, i.e. 80% of participants reported some improvement, and 20% reported a slightly worse condition.

### CONCLUSIONS

The results from Example 1 found an 83 % improvement rate, with rmost success in the eczema group of customers.

The results for Example 2 found between 80% and 100% of participants in the trial reporting improvements. In only two groups did any participants report the condition becoming worse. In FIG. 1A 3% reported a slight worsening of the condition, whereas in FIG. 1F (the child volunteer sample) 20% reported a slight worsening of the condition.

### DETAILED DESCRIPTION OF THE PREFERRED EMBODIMENTS

Preferred examples of compositions according to the invention are described below:

A number of different compositions, each having a different use were formulated and tested. The table below shows the results.

TABLE 4

| | | Range of | Range of |
|---|---|---|---|
| | Concentration of | concentration of | concentration of |
| | Sodium Silicate | Sodium Silicate | Sodium Silicate |
| | providing most | providing good | providing effective |
| Composition Type | effective results | treatment results | treatment results |
| Face cream (ointment) | 1 mg/kg | 0.5 to 2 mg/kg | 0.1 to 5 mg/kg |
| Body cream (ointment) | 2 mg/kg | 0.5 to 7 mg/kg | 0.25 to 20 mg/kg |
| Hand cream (ointment) | 2 mg/kg | 0.5 to 7 mg/kg | 0.25 to 15 mg/kg |
| Foot cream (ointment) | 2 mg/kg | 0.5 to 7 mg/kg | 0.25 to 20 mg/kg |
| Body and foot spray | 2 mg/kg | 0.5 to 7 mg/kg | 0.25 to 20 mg/kg |
| Shampoo for scalp | 1 mg/kg | 0.5 to 2 mg/kg | 0.1 to 5 mg/kg |
| Sun Screen cream and | 1 mg/kg | 0.5 to 2 mg/kg | 0.1 to 5 mg/kg |
| spray | | | |
| Shaving cream for face | 1 mg/kg | 0.5 to 2 mg/kg | 0.1 to 5 mg/kg |
| and body | | | |

The face, body, hand and foot creams included other substances, namely:

Water, Glycerine, Aloe Barbadensis, Tetrasodium EDTA, Methlyparaben, Paraffinum Liquidum, Triticum Vulgare, Glyceryl Stearate, Elaesis Guineensis, Cetyl Alcohol, Propylparaben, Benzyl Alcohol, Citric acid, natural palm oil, and an effective amount of sodium silicate.

TABLE 5
Examples of cream and emollient formulations
are set out in the table below:

| | Body/Hand/Foot | | Sunscreen |
|---|---|---|---|
| Component | Cream | Emollient | Cream |
| Glycerine | 7.00 | 7.00 | 7.00 |
| Aloe Vera Gel | 6.00 | 6.00 | 6.00 |
| Light Liquid Paraffin | 8.00 | 8.00 | 8.00 |
| Wheatgerm Oil | 4.00 | 4.00 | 4.00 |
| Palm Oil | 2.50 | 2.50 | 2.50 |
| Glceryl Stearate | 7.00 | 7.00 | 7.00 |
| Grapeseed Extract | 0.02 | 0.02 | 0.02 |
| Sodium Silicate | 0.002 | 0.001 | 0.001 |
| Vitamin E | 1.00 | 1.00 | 1.00 |
| Vitamin C | 1.00 | 1.00 | 1.00 |
| Jojoba | 2.00 | 2.00 | 2.00 |
| Tetrasodium EDTA | 0.20 | 0.20 | 0.20 |
| Soft White Paraffin | | 8.00 | 8.00 |
| Luteine | | | 5.00 |
| Zinc Oxide Micronised | | | 3.00 |
| Avobenzone (Parsol | | | 1.50 |
| 1789) | | | |
| Octyl | | | 2.00 |
| Methoxycinnamate | | | |
| Octyl Salicylate | | | 2.00 |
| Tricontanyl PVP | | | 2.00 |
| Water | to 100% | to 100% | to 100% |

Oitments (Emollients) were similar to creams but thicker in viscosity.

A body spray was formulated using the same constituents as for the body cream, but with each constituent diluted to 20% of the amount shown in the table. Purified water was added to rriake the body spray up to 100%.

Benzyl Alcohol, Citric acid, natural palm oil, and an effective amount of sodium silicate.

TABLE 6
Another example of a cream formulation
is set out in the table below:

| Component | INIC name | Body/Hand/Foot Cream |
|---|---|---|
| Glycerine | Glycerine | 7.00 |
| Aloe Vera Gel | Aloe Barbadensis | 5.00 |
| Light Liquid Paraffin | Paraffinum Liquidum | 8.00 |
| Wheatgerm Oil | Triticum Vulgar | 4.00 |
| Palm Oil | Elaeis Guineensis | 2.50 |
| Glceryl Stearate | Glyceryl Stearate | 6.00 |
| Grapeseed Extract | Vitrus Vinifera | 0.02 |
| Sodium Silicate | Sodium Silicate | 0.002 |
| Vitamin E | Vitamin E Acetate | 1.00 |
| Vitamin C | Sodium Ascorbyl | 1.00 |
| | Phosphate | |
| Tetrasodium EDTA | Tetrasodium EDTA | 0.20 |
| Alkoline GC | Hydrogenated | 0.50 |
| | Vegetable Glyerides | |
| | Citrate | |
| Alkoline SL | Sodium Stearoyl | 0.25 |
| | Lactylae | |
| Jojoba Oil | Buxua Chimenesis | 3.00 |
| Phenonip | Methylparaben | 0.12 |
| Phenonip | Propylparaben | 0.015 |
| Phenonip | Ethylyparaben | 0.030 |
| Phenonip | Butylparaben | 0.030 |
| Phenonip | Isobutyparaben | 0.015 |
| Phenonip | Phenoxyethanol | 0.540 |
| Water | Aqua | to 100% |

1. A composition comprising sodium silicate for the treatment of a dry skin condition, wherein the proportion of sodium silicate included in the composition is in the range of 0.1 to 20 mg/kg.

2. (canceled)

3. A composition according to claim 1, wherein the dry skin condition is one of eczema, dermatitis, psoariasis, acne, acne rosacea, cradle cap, dermatophyte infections, external vaginal thrush, facial malar rash due to systemic lupus erythmatosis.

4. A composition according to claim 1, wherein sodium silicate is included in the composition in the range of 0.5 to 7 mg/kg.

5. A composition according to claim 4, wherein sodium silicate is included in the composition in the amount of 1 mg/kg.

6. A composition according to claim 4, wherein sodium silicate is included in the composition in the amount of 2 mg/kg.

7. A composition according to claim 1 in the form of a cream.

8. A composition according to claim 7, wherein the cream is a face cream.

9. A composition according to claim 7, wherein the cream is a body cream.

10. A composition according to claim 7, wherein the cream is a foot cream.

11. A composition according to claim 7, wherein the cream is a shaving cream.

12. A composition according to claim 7, wherein the cream is a nail cream.

13. A composition according to claim 1, in the form of a spray.

14. A composition according to claim 1, in the form of a shampoo for the treatment of dry skin conditions affecting the scalp.

15. A composition according to claim 1, in the form of an ointment.

16. A composition according to claim 7, wherein the cream comprises glycerine, aloe vera gel, light liquid paraffin, wheatgerm oil, palm oil, glyceryl stearate, grapeseed extract, sodium silicate, vitamin E, vitamin C, jojoba, tetrasodium EDTA, and water.

17. A composition according to claim 16, wherein the cream comprises glycerine 7.00%, aloe vera gel 6.00%, light liquid paraffin 8.00%, wheatgerm oil 4.00%, palm oil 2.50%, glyceryl stearate 7.00%, grapeseed extract 0.02%, sodium silicate 0.002%, vitamin E 1.00%, vitamin C 1.00%, jojoba 2.00%, tetrasodium EDTA 0.20% and water to 100%.

18. A composition according to claim 16, wherein the cream comprises glycerine 7.00%, aloe vera gel 5.00%, light liquid paraffin 8.00%, wheatgerm oil 4.00%, palm oil 2.50%, glyceryl stearate 6.00%, grapeseed extract 0.02%, sodium silicate 0.002%, vitamin E 1.00%, vitamin C 1.00%, jojoba 2.00%, tetrasodium EDTA 0.20% and water to 100%.

19. A composition according to claim 16, further comprising at least one of the group consisting of alkaline gc, alkaline sl, methylparaben, propylparaben, ethylparaben, butylparaben, isobutylparaben, and phenoxyethanol.

20. A composition according to claim 19, wherein the cream comprises alkaline gc 0.50%, alkaline sl 0.25%, methylparaben 0.12%, propylparaben 0.015%, ethylparaben 0.030%, butylparaben 0.030%, isobutylparaben 0.015%, and phenoxyethanol 0.540%.

21. A composition according to claim 7, wherein the cream is a sun cream and further comprises sun blocking agents.

22. A composition according to claim 21, wherein the sun cream includes soft white paraffm, luteine, zinc oxide, avobenzone, octyl methoxycinnamate, octyl salicylate, tricontanyl PVP.

23. A composition according to claim 21, wherein the sun cream comprises glycerine 7.00%, aloe vera gel 6.00%, light liquid paraffin 8.00%, wheatgerm oil 4.00%, palm oil 2.50%, glyceryl stearate 7.00%, grapeseed extract 0.02%, sodium silicate 0.002%, vitamin E 1.00%, vitamin C 1.00%, jojoba 2.00%, tetrasodium EDTA 0.20%, soft white paraffin 8.00%, luteine 5.00%, zinc oxide 3.00%, avobenzone 1.50%, octylmethoxycinnamate 2.00%, octyl salicylate 2.00%, tricontanyl PVP 2.00% and water to 100%.

24. A composition according to claim 15, wherein the ointment comprises glycerine, aloe vera gel, light liquid paraffin, wheatgerm oil, palm oil, glyceryl stearate, grapeseed extract, sodium silicate, vitamin E, vitamin C, jojoba, tetrasodium EDTA, soft white paraffin, and water.

25. A composition according to claim 24, wherein the ointment comprises glycerine 7.00%, aloe vera gel 6.00%, light liquid paraffin 8.00%, wheatgerm oil 4.00%, palm oil 2.50%, glyceryl stearate 7.00%, grapeseed extract 0.02%, sodium silicate 0.002%, vitamin E 1.00%, vitamin C 1.00%, jojoba 2.00%, tetrasodium EDTA 0.20%, soft white paraffin 8.00%.

26. A method of treating dry skin conditions comprising the step of applying a composition as claimed in claim 1 to an area of a human or animal body affected by a dry skin condition.

27. Use of a composition comprising sodium silicate for the manufacture of a medicament for the treatment of a dry skin condition, wherein the proportion of sodium silicate included in the composition in the range of 0.1 to 20 mg/kg.
